(19) United States
(12) Patent Application Publication
(10) Pub. No.: US 2009/0053165 A1
(43) Pub. Date: Feb. 26, 2009

(54) Method for Treating Damaged Hair

(76) Inventors: Mark Anthony Brown, Union, KY (US); Thomas Allen Hutchins, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/131,060

(22) Filed: May 31, 2008

Related U.S. Application Data
(63) Continuation-in-part of application No. 11/894,144, filed on Aug. 20, 2007.
Application No. 11/894,144 is a continuation-in-part of application No. 11/981,036, filed on Oct. 31, 2007.

(51) Int. Cl. A61K 31/74; A61Q 5/00; G06Q 30/00

(52) U.S. Cl. 424/78.08; 705/14

(57) ABSTRACT

The present invention relates to a method for treating damaged hair comprising the steps of: a) contacting the hair with a shampoo composition, the shampoo comprising: from about 5% to about 50% of an anionic surfactant; from about 0.025% to about 5% by weight of a synthetic cationic polymer having a cationic charge density from about 2 meq/gm to about 7 meq/gm, wherein the synthetic cationic polymer forms lyotropic liquid crystals upon combination with the anionic surfactant; and water; and b) rinsing the composition from the hair, where, following the treatment, silicone deposition efficiency (DE) on the hair is greater than 1.

### CROSS REFERENCE TO RELATED APPLICATIONS

This application is a continuation-in-part of prior U.S. application Ser. No. 11/894,144, filed Aug. 20, 2007 and U.S. application Ser. No. 11/981,036, filed Oct. 31, 2007.

### FIELD OF THE INVENTION

The present invention relates to a method for treating damaged hair by applying a shampoo composition which contains lyotropic liquid crystals to the hair, as well as to articles of manufacture comprising the shampoo composition, and methods of promoting the sale thereof.

### BACKGROUND OF THE INVENTION

Hair can suffer damage from a number of sources, such as environmental exposure to ultraviolet radiation and chlorine, chemical treatment, i.e., bleaching, coloring, perming, as well as mechanical influences, i.e., prolonged use of heated styling appliances.

Each hair fiber comprises two fundamental layers, the cortex, which provides the hair's strength and contains the bulk of keratin protein, and the cuticle, which protects the cortex fibers from damage and is responsible for the surface properties of hair, such as shine and smoothness. The outside surface of the hair cuticle is covered by a thin, protective, hydrophobic layer, the F-layer, which protects the hair against water and friction and contributes to hair's natural smooth and lubricious feel. When healthy, the hair cuticle reflects light on its surface, resulting in a visual shine. When environmental exposures, chemical treatments, or mechanical influences damage the cuticle and strip away its protective F-layer, the hair becomes increasingly hydrophilic, feels drier and rougher, and is more susceptible to further damage, including breakage and frayed or split ends.

It is known to use hair conditioners to treat damaged hair. More specifically, post-shampoo application of hair conditioners, such as leave-on or rinse-off products, is known and hair conditioning shampoos, which cleanse and condition the hair, are also known. Polydimethylsiloxanes (PDMS) are often employed as conditioning materials in both shampoo and conditioner applications to improve hair feel. It is known, however, that, in the case of more hydrophilic, damaged hair, PDMS deposition is greatly reduced and cannot provide the same benefit in hair condition as for undamaged or virgin hair.

Based on the foregoing, there is a need for a method for treating damaged hair to increase silicone deposition efficiency (DE), as defined below, on the hair, such that following treatment, DE on the hair is greater than 1. Furthermore, there is a need for a method of communicating to a consumer the ability of a shampoo composition to treat damaged hair and increase silicone deposition efficiency on the hair.

### SUMMARY OF THE INVENTION

The present invention relates to a method of treating damaged hair, comprising the steps of:
a) contacting said hair with a shampoo composition, said shampoo composition comprising:
   i from about 5% to about 50% of an anionic surfactant;
   ii from about 0.025% to about 5% by weight of a synthetic cationic polymer having a cationic charge density of from about 2 meq/gm to about 7 meq/gm, wherein said synthetic cationic polymer forms lyotropic liquid crystals upon combination with said anionic surfactant; and
   iii water; and
b) rinsing said composition from said hair
   wherein following said treatment, silicone deposition efficiency on said hair is greater than 1.

The present invention also relates to an article of manufacture useful for treating damaged hair, comprising (a) a package; (b) said package containing a shampoo composition, wherein said shampoo composition comprises lyotropic liquid crystals; and (c) information to communicate to consumers the ability of the shampoo composition to treat damaged hair.

The present invention also relates to methods of promoting the sale of shampoo compositions which are useful for treating damaged hair. The methods include a variety of steps to inform a consumer of the ability of the present shampoo compositions and articles to treat damaged hair and encourage the consumer to use them to treat damaged hair.

### DETAILED DESCRIPTION OF THE INVENTION

It has been discovered that compositions which form lyotropic liquid crystals are particularly useful in treating damaged hair.

A liquid crystalline state exists structurally between the solid crystalline phase and the liquid phase (i.e. an intermediate between the three dimensionally ordered crystalline state and the completely disordered liquid state).

The term “liquid crystal”, as used herein, means a material having phases that are ordered and/or crystalline in only one or two of their three possible orthogonal directions and are disordered (random and/or liquid-like ) in the other dimensions.

The term “lyotropic”, as used herein, means that the ordering effects of a material are induced by changing both its concentration and temperature.

The term “nonvolatile” refers to any material having little or no significant vapor pressure under ambient conditions, and a boiling point under one atmosphere (atm) preferably at least about 250° C. The vapor pressure under such conditions is preferably less than about 0.2 mm.

The term “polymer”, as used herein, shall include materials whether made by polymerization of one type of monomer or made by two (i.e., copolymers) or more types of monomers.

The term “water soluble”, as used herein, means that the polymer is soluble in water in the present composition. In general, the polymer should be soluble at 25° C. at a concentration of 0.1% by weight of the water solvent, preferably at 1%, more preferably at 5%, more preferably at 15%.

The term “damaged hair”, as used herein, includes moderately damaged hair, as available from International Hair Importers & Products Inc. under the code PGMDST, and hair of a condition similar to PGMDST hair, bleached hair, permed hair, and color-treated hair.

All percentages, parts and ratios are based upon the total weight of the compositions of the present invention, unless otherwise specified. All such weights as they pertain to listed ingredients are based on the active level and therefore do not include solvents or by-products that may be included in commercially available materials, unless otherwise specified.

All molecular weights as used herein are weight average molecular weights expressed as grams/mole, unless otherwise specified.

### Shampoo Composition

As discussed hereinbefore, the present invention includes a shampoo composition which contains lyotropic liquid crystals. The liquid crystals may form upon combination of the detersive surfactant component and cationic polymer discussed hereinafter. It has been discovered that application of the shampoo composition described herein to damaged hair increases the hydrophobicity of the hair and restores its natural smooth, lubricious feel.

Anionic Surfactant Component

The shampoo compositions for treating damaged hair comprise an anionic detersive surfactant component to provide cleaning performance to the composition and to aid in formation of the lyotropic liquid crystalline phase. The anionic surfactant component comprises an anionic detersive surfactant, and optionally, a zwitterionic and/or amphoteric detersive surfactant, which has an attached group that is anionic at the pH of the composition. Such surfactants should be physically and chemically compatible with the essential components described herein or should not otherwise unduly impair product stability, aesthetics, or performance.

Suitable anionic detersive surfactant components include those which are known for use in hair care or other shampoo cleansing compositions. The concentration of the anionic surfactant component generally ranges from about 5% to about 50%, preferably from about 8% to about 30%, more preferably from about 10% to about 25%, even more preferably from about 12% to about 20%, by weight of the composition.

Preferred anionic detersive surfactants for use in the shampoo compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Suitable amphoteric or zwitterionic detersive surfactants for use in the shampoo composition herein include those which are known for use in hair care or other personal care cleansing compositions and those which contain a group that is anionic at the pH of the shampoo composition. The concentration of such amphoteric detersive surfactants preferably ranges from about 0.5% to about 20%, preferably from about 1% to about 10% by weight of the composition. Non-limiting examples of suitable zwitterionic or amphoteric surfactants are described in U.S. Pat. Nos. 5,104,646 and 5,106,609.

The shampoo compositions may further comprise additional surfactants for use in combination with the anionic detersive surfactant component described hereinbefore. Suitable optional surfactants include nonionic surfactants, cationic surfactants, and combinations thereof. Any such surfactant known in the art for use in hair or personal care products may be used, provided that the optional additional surfactant is also chemically and physically compatible with the essential components of the shampoo composition or does not otherwise unduly impair product performance, aesthetics or stability. The concentration of optional additional surfactants in the shampoo composition may vary with the cleansing or lather performance desired, the optional surfactant selected, the desired product concentration, the presence of other components in the composition, and other factors well known in the art.

Non limiting examples of other anionic, zwitterionic, amphoteric, or optional additional surfactants suitable for use in the shampoo compositions are described in U.S. Pat. Nos. 3,929,678; 2,658,072; 2,438,091; and 2,528,378.

Synthetic Cationic Polymer

The cationic polymer described herein aids in providing damaged hair with a surrogate hydrophobic F-layer. The microscopically thin F-layer provides natural weatherproofing, while helping to seal in moisture and prevent further damage. Environmental exposures, chemical treatments, and mechanical influences damage the hair cuticle and strip away its protective F-layer. As the F-layer is stripped away, the hair becomes increasingly hydrophilic, thereby reducing silicone deposition on the hair (as discussed above). It has been found that when lyotropic liquid crystals are applied to damaged hair, the hair becomes more hydrophobic and more virgin-like, in both look and feel. Without being limited to any theory, it is believed that the lyotropic liquid crystal complex creates a surrogate hydrophobic layer or film, which coats the damaged hair fibers and protects the hair, much like the natural F-layer protects the hair. Silicone deposits more readily on this surrogate film than on the hydrophilic surface of (untreated) damaged hair. The application of a liquid crystal shampoo on the surface of a damaged hair fiber increases silicone deposition efficiency (DE), as defined below, on the hair, such that DE is greater than 1.

Lyotropic liquid crystals are formed by combining the synthetic cationic polymers described herein with the aforementioned anionic detersive surfactant component of the shampoo composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable shampoo composition that provides improved conditioning performance, with respect to damaged hair. In some embodiments, the synthetic cationic polymer may be formed from

i) one or more cationic monomer units, and optionally

ii) one or more momomer units bearing a negative charge, and/or

iii) a nonionic momomer,

wherein the subsequent charge of the copolymer is positive. The ratio of the three types of monomers is given by “m”, “p” and “q” where “m” is the number of cationic monomers, “p” is the number of momomers bearing a negative charge and “q” is the number of nonionic momomers.

The concentration of the cationic polymers ranges about 0.025% to about 5%, preferably from about 0.1% to about 3%, more preferably from about 0.2% to about 1%, by weight of the shampoo composition.

The cationic polymers have a cationic charge density of from about 2 meq/gm to about 7 meq/gm, preferably from about 3 meq/gm to about 7 meq/gm, more preferably from about 4 meq/gm to about 7 meq/gm. In some embodiments, the cationic charge density is about 6.2 meq/gm. The polymers also have a molecular weight of from about 1,000 to about 5,000,000, more preferably from about 10,000 to about 2,000,000, most preferably 100,000 to about 2,000,000.

In one embodiment, the cationic polymers are water soluble or dispersible, non-crosslinked, synthetic cationic polymers having the following structure:

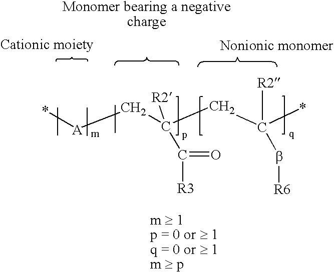

Where A, may be one or more of the following cationic moieties:

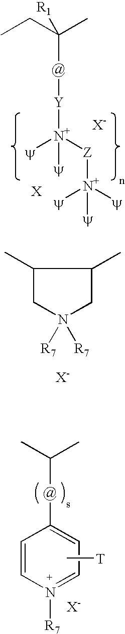

Where @=amido, alkylamido, ester, ether, alkyl or alkylaryl.
Where Y=C1-C22 alkyl, alkoxy, alkylidene, alkyl or aryloxy.
Where Ψ=C1-C22 alkyl, alkyloxy, alkyl aryl or alkyl aryloxy.
Where Z=C1-C22 alkyl, alkyloxy, aryl or aryloxy.
Where R1=H, C1-C4 linear or branched alkyl.
Where s=0 or 1 , n=0 or ≧1.
Where T and R7=C1-C22 alkyl.
Where X−=halogen, hydroxide, alkoxide, sulfate or alkylsulfate.

Where the monomer bearing a negative charge is defined by R2′=H, C1-C4 linear or branched alkyl and R3 as:

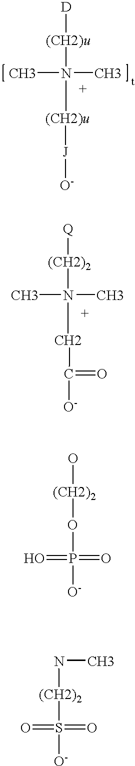

Where D=O, N, or S.
Where Q=NH₂ or O.
Where u=1-6.
Where t=0-1.
Where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

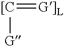

Where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide, ethylenimine, vinylamine, 2-vinylpyridine, 4-vinylpyridine, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride, diallyldimethyl ammonium chloride.

Preferred cationic monomers comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Preferred cationic monomers include trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride.

More preferred cationic monomers include trimethyl ammonium propyl(meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Preferred monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated(meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Preferred nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X−) in association with the synthetic cationic polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

Optional Ingredients

The shampoo composition may further comprise optional ingredients selected from the group consisting of oily conditioning agents, hydrocarbon oils, polyolefins, fatty esters, fluorinated conditioning compounds, fatty alcohols, quaternary ammonium compounds, polyethylene glycols, anti-dandruff actives, anti-microbial actives, inorganic or synthetic particles, opacifying agents, suspending agents, propellants, paraffinic hydrocarbons, mono or divalent salts, fragrances, vitamins, chelating agents, colorants, pigments, dyes, phase separation initiators such as electrolytes, and mixtures thereof. These optional components are described in detail in U.S. Patent Publication No. 2003/0223951A1. Suspending agents are described in U.S. Pat. No. 5,756,436. Such optional ingredients may be present in an amount of from about 0.1% to about 5% by weight of the shampoo composition.

Silicone Conditioning Agent

If an oily conditioning agent is included, it is preferably in the form of a water-insoluble silicone conditioning agent. The silicone conditioning agent may comprise volatile silicone, non-volatile silicone, or combinations thereof. Non-volatile silicone conditioning agents are preferred. If volatile silicones are present, their presence is typically incidental to their use as a solvent or carrier for commercially available forms of non-volatile silicone materials, such as silicone gums and resins. The silicone conditioning agent particle may be in the form of a silicone resin, or it may be in the form of a silicone fluid (ie. dimethicone droplets).

Non-limiting examples of suitable silicone conditioning agents and optional suspending agents for the silicone are described in U.S. Reissue Pat. No. 34,584, U.S. Pat. Nos. 5,104,646, and 5,106,609. The silicone conditioning agents for use in the compositions preferably have a viscosity, as measured at 25° C., of from about 20 to about 2,000,000 centistokes (“csk”), more preferably from about 1,000 to about 1,800,000 csk, even more preferably from about 5,000 to about 1,500,000 csk, more preferably from about 10,000 to about 1,000,000 csk.

The oil droplets preferably have a volume average particle diameter of from about 0.01 microns to about 100 microns. For small particle application to hair, the volume average particle diameters preferably range from about 0.01 microns to about 4 microns, more preferably from about 0.01 to about 2 microns, even more preferably from about 0.01 microns to about 0.5 microns. For larger particle application to hair, the volume average particle diameters preferably range from about 4 microns to about 50 microns, more preferably from about 9 microns to about 45 microns, even more preferably from about 25 microns to about 40 microns, still more preferably from about 25 microns to about 35 microns. The more preferred range of about 25 microns to about 35 microns maximizes silicone deposition efficiency and shampoo phase stability. Particle size is measured using the LA-910 Particle Size Analyzer, manufactured by Horiba.

Non-volatile silicone oils suitable for use in the compositions may be selected from organo-modified silicones and fluoro-modified silicones. In one embodiment, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups.

In a preferred embodiment, the non-volatile silicone oil is polydimethylsiloxane.

Silicone fluids suitable for use in the compositions are disclosed in U.S. Pat. Nos. 2,826,551; 3,964,500; and 4,364,837, British Patent No 849,433, and Silicon Compounds, Petrarch Systems, Inc. (1984).

Silicone Deposition Efficiency

The present methods of treating damaged hair increase silicone deposition efficiency (DE), as defined below, on the hair, such that following treatment, DE on the hair is greater than 1. Silicone deposition efficiency (DE) it generally less than 1000, typically less than 800, more typically less than 600, preferably less than 400, more preferably less than 200.

The deposition efficiency (DE) of silicone is calculated as follows:

$$\frac{( {P}_{D} / {P}_{V} )}{( ( {C}_{D} + 1 ) / {C}_{V} )} = D E ,$$

where P_{D}=amount of silicone (ppm) deposited on damaged hair that has been treated with a shampoo composition comprising a cationic polymer; P_{V}=amount of silicone (ppm) deposited on virgin hair that has been treated with a shampoo composition comprising a cationic polymer; C_{D}=amount of silicone (ppm) deposited on damaged hair that has been treated with a cationic-polymer-free shampoo composition; C_{V}=amount of silicone (ppm) deposited on virgin hair that has been treated with a cationic-polymer-free shampoo composition.

Of note, the shampoo composition comprising a cationic polymer (P) and the cationic-polymer-free shampoo composition (C) may or may not contain a silicone. In other words, for purposes of determining the silicone deposition efficiency (DE) of a shampoo composition comprising a cationic polymer, the source of the silicone may be the shampoo composition itself or a separate composition, i.e., a conditioning composition, which is applied after the shampoo composition is applied. Similarly, with regard to the cationic-polymer-free shampoo and the variables C_{D} and C_{V}, the source of the silicone may be the cationic-polymer-free shampoo itself or a separate composition, i.e., a conditioning composition, which is applied after the cationic-polymer-free shampoo composition is applied. For example, in the sample calculation that follows, as well as in the enclosed data (for examples #5, #11, #15, #20, and #21), the shampoo composition comprising a cationic polymer (composition of example #5) and the cationic-polymer-free shampoo (composition of example #21) both contain silicone.

Sample Calculation (P_{D}=amount of silicone (ppm) deposited by the shampoo composition of example #5 on bleached hair; P_{V}=amount of silicone (ppm) deposited by the shampoo composition of example #5 on virgin hair; C_{D}=amount of silicone (ppm) deposited by the shampoo composition of example #21 on bleached hair; C_{V}=amount of silicone (ppm) deposited by the shampoo composition of example #21 on virgin hair):

$$\begin{matrix}\frac{( {P}_{D} / {P}_{V} )}{( ( {C}_{D} + 1 ) / {C}_{V} )} = \frac{( 353 ppm / 648 ppm )}{( ( 0 ppm + 1 ppm ) / 465 ppm )} \\ = 253\end{matrix}$$

In certain embodiments of the present invention, following treatment with a shampoo composition comprising a cationic polymer, the silicone deposition efficiency (DE) on moderately damaged hair is greater than 1. In some embodiments of the invention, following treatment, the silicone deposition efficiency (DE) on permed hair is greater than 1. In further embodiments, following treatment, the silicone deposition efficiency (DE) on bleached hair is greater than 1. In certain embodiments, following treatment, the silicone deposition efficiency (DE) on color-treated hair is greater than 1. Preferably, following treatment, the silicone deposition efficiency (DE) on damaged hair, including moderately damaged hair, permed hair, bleached hair, and color-treated hair, is greater than 1.

### Method for Treating Damaged Hair

The compositions described herein are particularly useful in treating damaged hair. Damaged hair includes hair that has been exposed to environmental damage, such as damage from ultraviolet radiation and chlorine, chemical treated hair, i.e., bleached, color-treated, and/or permed hair, as well as mechanically damaged hair, i.e., hair exposed to prolonged use of heated styling appliances. As discussed above, such hair is increasingly hydrophilic (increased surface energy), as compared to virgin hair.

The method of treating damaged hair comprises the steps of contacting damaged hair, which has preferably been wetted with water, with an effective amount of the shampoo composition described herein. After contacting the hair with the shampoo composition, the composition is rinsed from the hair. Effective amounts of the shampoo composition generally range from about 1 gm to about 50 gm, preferably from about 1 gm to about 20 gm. Application to the hair typically includes working the composition through the hair such that most or all of the hair is contacted with the composition.

### Article of Manufacture for Treating Damaged Hair

The present invention also relates to an article of manufacture useful for treating damaged hair. The article comprises a package which contains the shampoo composition described herein. The package is in association with information or instructions, in the form of indicia, which informs the consumer that the shampoo composition will treat and improve the quality of damaged hair, i.e., provide a more lubricious feel and increased shine to the hair. The indicia may be in the form of words, pictures, symbols, or the like. Furthermore, the package may include a claim of superiority over other shampoo compositions. As used herein, the phrase “in association with” means the information or instructions are either directly printed on the package itself or presented in a different manner, including, but not limited to, as promotional material to communicate the information or instructions to a consumer. The information or instructions are important to encourage consumers, especially those with chemically treated hair, to use the shampoo composition described herein.

In another embodiment, the package may bear information that informs the consumer that the shampoo composition provides one or more benefit selected from increasing silicone deposition efficiency on the hair, providing hair with a protective hydrophobic layer, restoring hair to a virgin-like state, restoring hair shine, or combinations thereof The lyotropic liquid crystals present in the shampoo compositions herein have been found to provide these and other benefits to damaged hair.

The package should be any package suitable for containing liquid compositions. In the case of shampoo compositions, such packages are typically formed from petroleum-based plastics such as PET.

### Method of Promoting the Sale of Article of Manufacture for Treating Damaged Hair

The present invention also relates to methods for promoting the sale of the aforementioned articles of manufacture. The present methods generally comprise providing promotional materials to consumers by a variety of steps to inform them of the benefits of the present shampoo compositions for damaged hair and particularly to communicate the function of lyotropic liquid crystals in treating damaged hair.

In one embodiment, the method comprises promoting the sale of a shampoo product which contains lyotropic liquid crystals, comprising the steps of (a) displaying, shelving, or merchandising the shampoo product in a retail store; and/or (b) providing promotional materials to consumers, wherein said promotional materials comprise information regarding the shampoo product's ability to treat damaged hair and/or an instruction to apply the shampoo product to hair which has been chemically treated, exposed to environmental damage, or mechanically damaged.

In another embodiment, the method includes sending promotional materials directly to consumers via mail or electronic mail. The promotional materials can also include samples of the shampoo compositions herein, or articles, and can include discount coupons, which the consumer can redeem upon purchasing the present shampoo compositions or articles.

In yet another embodiment, the method includes providing promotional materials to a hair styling salon, which is intended to encourage the stylist to provide the promotional materials or information to his or her customers, preferably to customers with damaged hair. For example, a consumer with chemically treated hair may have the chemical treatment performed by a professional stylist. It is believed that such stylists would be enabled to effectively communicate the benefits of lyotropic liquid crystals to customers—preferably customers with damaged hair—upon receipt and review of the promotional materials of the present invention.

### Method of Manufacture

The compositions, in general, may be made by mixing together at elevated temperature, e.g., about 72° C., water and surfactants along with any solids (e.g. amphiphiles) that need to be melted, to speed mixing into the personal cleansing composition. The ingredients are mixed thoroughly at the elevated temperature and then cooled to ambient temperature. Additional ingredients, including electrolytes, polymers, and particles, may be added to the cooled product. The silicone may be emulsified at room temperature in concentrated surfactant and then added to the cooled product.

All exemplified amounts are listed as weight percents and exclude minor materials such as diluents, preservatives, color solutions, imagery or conceptual ingredients, botanicals, and so forth, unless otherwise specified.

### Testing Methods

The following procedures were used to evaluate the compositions of the invention. Specifically, silicone deposition is measured according to the method described below and then used to calculate silicone deposition efficiency, according to the equation described above. Relative to the data below, the evaluated hair samples (“switches”) are prepared or obtained according to the following techniques.

Preparation of Damaged Hair Switches:

Virgin (commonly referred to as special quality hair or special quality virgin hair) hair, moderately damaged hair, and bleached hair (commonly referred to as low lift substrate hair) are purchased from International Hair Importers & Products Inc., 87-29 Myrtle Ave., Glendale, N.Y. 11385, under the codes SPQ, for the virgin hair, SPQLLS, for the bleached hair, and PGMDST, for the moderately damaged hair.

Permed hair is prepared using commercially available Option 1™ Perm (Innovative Styling Options, Inc., Darien, Conn.). 4-gram, 8-inch switches of virgin hair are first rinsed, with water, for about 30 seconds. After rinsing, the switches are sandwiched between one's index and middle fingers and pulled through the fingers to remove excess water. Each switch is then blotted gently with a paper towel to dry the switch. The switches are then placed on a sheet of plastic wrap on a tray and a syringe is used to apply 0.10 cubic centimeters (cc) of Option1™ Prewrap to each switch. The Prewrap is then worked into each hair switch. Then, 2.0 cc of Option 1™ Waving Lotion is applied to each switch and gently worked through each switch. The treated switches are then left to rest at room temperature for about 20 minutes. Afterwards, the switches are rinsed with water, for about 30 seconds. After rinsing, the switches are sandwiched between one's index and middle fingers and pulled through the fingers to remove excess water. Each switch is then blotted gently with a paper towel to dry the switch. The switches are then placed on a clean sheet of plastic wrap on a tray. Next, 2.0 cc of Option 1™ Neutralizer solution is applied with a syringe to each switch. The treated switches are then left to rest at room temperature for about 5 minutes. Afterwards, the switches are rinsed with water, for about 30 seconds. Excess water is removed from the rinsed switches and the switches are hung to dry. Water used for rinsing hair switches is typically at a temperature of about 100° F. and a pressure of about 1.5 gal/min.

Color-treated hair is prepared using commercially available Nice n' Easy® hair color. 4-gram, 8-inch switches of bleached hair (purchased from International Hair Importers & Products Inc., 87-29 Myrtle Ave., Glendale, N.Y. 11385) are placed on a sheet of plastic wrap on a tray. The hair colorant is prepared according to the instructions provided with the colorant. 12 cc of colorant are applied with a syringe to the front side of each switch on the tray. The colorant is then massaged into each switch, for about 30 seconds per switch, making sure that the hair fibers are separated and that the colorant is applied evenly to each strand of hair. The switches are then turned over and 12 cc of colorant are applied to the back side of each switch. Again, the colorant is massaged or worked into the hair, for about 30 seconds per switch, i.e., by spreading the switch back and forth, making sure that the hair fibers are separated and that the colorant is applied evenly to each strand of hair. The treated switches are then wrapped in plastic wrap and placed in an oven, at about 30° C. to about 32° C., for about 30 minutes. Afterwards, the front sides of the switches are rinsed in water for about 1 minute, while running one's fingers through the hair. After rinsing, the switches are sandwiched between one's index and middle fingers and pulled through the fingers to remove excess water. The switches are then turned over and the back sides of the switches are rinsed in water for about 1 minute, while running one's fingers through the hair. After this rinse, the switches are sandwiched between one's index and middle fingers and pulled through the fingers to remove excess water. Water used for rinsing hair switches it typically at a temperature of about 100° F. and a pressure of about 1.5 gal/min.

The technique for treating the switches with a shampoo composition is discussed below.

Application of Shampoo Compositions to Hair Samples:

Hair switches are hung over a sink and pre-wetted with water for about 30 seconds. The switches are then sandwiched between one's index and middle fingers and pulled through the fingers to remove excess water. 0.4 cc of shampoo composition is applied to the front side of each hair switch, in a zig-zag manner down the length of each switch. The shampoo is brushed into each hair switch, for about 30 seconds, using a small, Goody®, stiff-bristle, plastic brush. Each hair switch is then rinsed with water for about 30 seconds. The switches are then sandwiched between one's index and middle fingers and pulled through the fingers to remove excess water. The hair switches are then flipped over and 0.4 cc of shampoo composition is applied to the back side of each hair switch, in a zig-zag manner down the length of each switch. Each hair switch is then rinsed with water for about 30 seconds. The switches are then sandwiched between one's index and middle fingers and pulled through the fingers to remove excess water. The hair switches are then air-dried. Water used for pre-wetting and rinsing hair switches is typically at a temperature of about 100° F. and a pressure of about 1.5 gal/min. The water is typically at a hardness of about 7 grains/gallon to about 13 grains/gallon.

The above-steps, with the exception of the pre-wetting step, are repeated four times for each hair switch (for a total of five applications). After the fifth application, silicone deposition on each hair switch is measured, according to the protocol that follows. Alternatively, if the shampoo composition applied according to the above protocol does not contain silicone, then a conditioning composition containing silicone is applied, after the fifth application of the shampoo composition, and silicone deposition is measured after the application of the conditioning composition.

In the data that follows, the hair switches are each treated with a different shampoo composition, represented in the tabulated data. One switch is treated with the shampoo composition of Example #15 below. Another hair switch is treated with the shampoo composition of Example #5 below. A third hair switch is treated with the shampoo composition of Example #11 below. A fourth hair switch is treated with the shampoo composition of Example #20 below. A final hair switch is treated with the shampoo of Comparative Example #21 Each of these compositions contains silicone. Treatment of the switches with shampoo composition involves the steps described above.

Measurement of Silicone Deposition and Calculation of Silicone Deposition Efficiency:

1.5 g of hair is placed in a 20 mL scintillation vial. 6 mL of 50:50 toluene:methylisobutyl ketone is added to the vial, in order to extract silicone from the hair. The vials are agitated on a pulsed vortexer for 30 minutes. The silicone in the extract is quantified using inductively coupled plasma optical emission spectrometry (ICP-OES). ICP calibration standards of known silicone concentration are made using the same or a structurally comparable type of silicone raw material as the products being tested. The working range of the method is about 8 to about 2300 ppm.

The tabulated readings below represent the average of 4 total readings, taken on 4 hair switches, each switch prepared and treated according to the techniques described above.

Silicone Deposition

| | | | Moderately | | Color- | |
|---|---|---|---|---|---|---|
| Silicone Deposition on Hair (ppm) | Example # | Virgin | Damaged | Permed | treated | Bleached |
| (30:70) AM:MAPTAC (4.0 meq/g, 227 meq/Å) | 15 | 1272 | 1419 | 763 | 276 | 416 |
| DADMAC (6.2 meq/g, 162 meq/Å) | 5 | 648 | 639 | 427 | 211 | 353 |
| (10:90) BEM:MAPTAC (2.6 meq/g, 292 meq/Å) | 11 | 541 | 482 | 353 | 153 | 201 |
| (50:50) AM:MAPTAC (3.4 meq/g, 162 meq/Å) | 20 | 386 | 404 | 145 | 56 | 87 |
| CATIONIC-POLYMER-FREE | 21 | 465 | 362 | 91 | 0* | 0* |

*Below lower detection limit of method (about 8 ppm)

Silicone Deposition Efficiency (DE)

| | | Moderately | | | | |
|---|---|---|---|---|---|---|
| Silicone Deposition on Hair (ppm) | Virgin | Damaged | Permed | Colored | Bleached | DE |
| (30:70) AM:MAPTAC (4.0 meq/g, 227 meq/Å) | 1272 | 1419 | | | | 1.43 |
| DADMAC (6.2 meq/g, 162 meq/Å) | 648 | 639 | | | | 1.26 |
| (10:90) BEM:MAPTAC (2.6 meq/g, 292 meq/Å) | 541 | 482 | | | | 1.14 |
| (50:50) AM:MAPTAC (3.4 meq/g, 162 meq/Å) | 386 | 404 | | | | 1.34 |
| CATIONIC-POLYMER-FREE | 465 | 362 | | | | 1.00 |
| (30:70) AM:MAPTAC (4.0 meq/g, 227 meq/Å) | 1272 | | 763 | | | 3.03 |
| DADMAC (6.2 meq/g, 162 meq/Å) | 648 | | 427 | | | 3.33 |
| (10:90) BEM:MAPTAC (2.6 meq/g, 292 meq/Å) | 541 | | 353 | | | 3.30 |
| (50:50) AM:MAPTAC (3.4 meq/g, 162 meq/Å) | 386 | | 145 | | | 1.90 |
| CATIONIC-POLYMER-FREE | 465 | | 91 | | | 1.00 |
| (30:70) AM:MAPTAC (4.0 meq/g, 227 meq/Å) | 1272 | | | 276 | | 101 |
| DADMAC (6.2 meq/g, 162 meq/Å) | 648 | | | 211 | | 151 |
| (10:90) BEM:MAPTAC (2.6 meq/g, 292 meq/Å) | 541 | | | 153 | | 131 |
| (50:50) AM:MAPTAC (3.4 meq/g, 162 meq/Å) | 386 | | | 56 | | 67.5 |
| CATIONIC-POLYMER-FREE | 465 | | | 0* | | 1.00 |
| (30:70) AM:MAPTAC (4.0 meq/g, 227 meq/Å) | 1272 | | | | 416 | 152 |
| DADMAC (6.2 meq/g, 162 meq/Å) | 648 | | | | 353 | 253 |
| (10:90) BEM:MAPTAC (2.6 meq/g, 292 meq/Å) | 541 | | | | 201 | 173 |
| (50:50) AM:MAPTAC (3.4 meq/g, 162 meq/Å) | 386 | | | | 87 | 105 |
| CATIONIC-POLYMER-FREE | 465 | | | | 0* | 1.00 |

*Below lower detection limit of method (about 8 ppm)

The following examples are representative of suitable shampoo compositions for use in the method of treating damaged hair according to the present invention. Also included are comparative examples of non-representative shampoo compositions.

### NON-LIMITING EXAMPLES

Hompolymers

| | EXAMPLE COMPOSITION | | | | | | | | | |
|---|---|---|---|---|---|---|---|---|---|---|
| | 1 | 2 | 3 | 4 | 5 | 6 | 7 | 8 | 9 | 10 |
| Ammonium Laureth Sulfate (AE₃S) | 6.50 | 6.00 | | | 6.00 | 7.50 | 7.50 | | | |
| Ammonium Lauryl Sulfate (ALS) | 8.10 | 10.00 | | | 10.00 | 6.50 | 6.50 | | | |
| Sodium Laureth Sulfate (SE₃S) | | | 6.00 | 6.50 | | | | 6.00 | 6.00 | 6.50 |
| Sodium Lauryl Sulfate (SLS) | 1.40 | | 10.00 | 5.50 | | | | 7.00 | 10.00 | 5.50 |
| Sodium Lauroamphoacetate⁽¹⁴⁾ | | | | | | | | | | 2.00 |
| Cocaminopropionic Acid⁽¹⁵⁾ | | | | 1.00 | | | | | | |
| Cocamidopropyl Betaine⁽¹⁶⁾ | | | | 1.00 | | | | 2.00 | | |
| Cocamide MEA | 1.00 | | | 0.80 | | 0.80 | 0.80 | 0.85 | | 0.80 |
| Cetyl Alcohol | 0.35 | | | 0.60 | | 0.60 | 0.60 | | | 0.60 |
| Lauryl Alcohol | 0.20 | | | 0.35 | | | | | | 0.35 |
| Laureth-4 Alcohol | | 0.90 | 0.90 | | 0.90 | | | | 0.90 | |
| Dihydrogenated Tallowamidoethyl | | | | 0.15 | | 0.15 | 0.15 | | | 0.15 |
| Hydroxyethylmonium Methosulfate⁽¹⁷⁾ | | | | | | | | | | |
| 1-Propanaminium, N,N,N-trimethyl-3- | 0.40⁽¹⁾ | | | | | | | | | |
| [(2-methyl-1-oxo-2-propenyl)amino]-, | | | | | | | | | | |
| chloride; | | | | | | | | | | |
| (Poly(Methacrylamidopropyl trimethyl | | | | | | | | | | |
| ammonium chloride))^{(1,2)} | | | | | | | | | | |
| Methacryloamidopropyl-pentamethyl- | | | | | | 0.40 | | | | |
| 1,3-propylene-2-ol-ammonium | | | | | | | | | | |
| dichloride⁽³⁾ | | | | | | | | | | |
| N,N,N,N′,N′,N″,N″-heptamethyl-N″-3- | | | | | | | 0.40 | | | |
| (1-oxo-2-methyl-2- | | | | | | | | | | |
| propenyl)aminopropyl-9- | | | | | | | | | | |
| oxo-8-azo-decane-1,4,10-triammonium | | | | | | | | | | |
| trichloride⁽¹⁸⁾ | | | | | | | | | | |
| diallyldimethyl ammonium chloride^{(4,5)} | | 0.10⁽⁵⁾ | 0.25⁽⁵⁾ | 0.50⁽⁴⁾ | 0.25⁽⁵⁾ | | | 0.10⁽⁵⁾ | 0.25⁽⁵⁾ | |
| [(2- | | | | | | | | | | 0.05 |
| methacryloyloxy)ethyl]trimethyl- | | | | | | | | | | |
| ammonium methylsulfate | | | | | | | | | | |
| homopolymer⁽⁶⁾ | | | | | | | | | | |
| Ethylene Glycol Distearate | | | 1.50 | 1.50 | | 1.50 | 1.50 | 1.50 | 1.50 | 1.50 |
| Trihydroxystearin⁽⁷⁾ | 0.25 | 0.10 | | | 0.10 | | | | | |
| Polyethylene Glycol (14000)⁽⁸⁾ | | | | 0.17 | | 0.17 | 0.17 | | | 0.17 |
| Fragrance | 0.55 | 0.60 | 0.65 | 0.55 | 0.60 | 0.55 | 0.55 | 0.65 | 0.65 | 0.55 |
| Sodium Chloride | 0.30 | 0.40 | 1.40 | 0.80 | 0.40 | 0.80 | 0.80 | 1.40 | 1.40 | 0.80 |
| Ammonium Xylenesulfonate | 0.20 | | | | | | | | | |
| Citric Acid | 0.04 | 0.04 | 0.22 | 0.22 | 0.04 | 0.04 | 0.04 | 0.22 | 0.22 | 0.22 |
| Sodium Citrate | 0.40 | 0.40 | | | 0.40 | 0.40 | 0.40 | | | |
| Sodium Benzoate | 0.25 | 0.25 | 0.25 | 0.25 | 0.25 | 0.25 | 0.25 | 0.25 | 0.25 | 0.25 |
| Ethylene Diamine Tetra Acetic Acid | 0.10 | 0.10 | 0.10 | 0.10 | 0.10 | 0.10 | 0.10 | 0.10 | 0.10 | 0.10 |
| Dimethicone^{(9,10,11)} | | 0.50⁽⁹⁾ | | 2.00⁽¹⁰⁾ | 1.00⁽⁹⁾ | 0.80⁽⁹⁾ | | 1.00⁽⁹⁾ | 2.00⁽⁹⁾ | 0.50⁽⁹⁾ |
| Polydecene⁽¹²⁾ | 0.40 | | | | | | | | | |
| Trimethylolpropane | 0.10 | | | | | | | | | |
| Tricaprylate/Tricaprate⁽¹³⁾ | | | | | | | | | | |
| Cosmetic Pigment (Mica, Titanium | | 0.10 | | | | | | | | |
| Dioxide)⁽¹⁹⁾ | | | | | | | | | | |
| Water and Minors (QS to 100%) | | | | | | | | | | |

Copolymers

| | EXAMPLE COMPOSITION | | | | | | | | | |
|---|---|---|---|---|---|---|---|---|---|---|
| | 11 | 12 | 13 | 14 | 15 | 16 | 17 | 18 | 19 | 20 |
| Ammonium Laureth Sulfate (AE₃S) | 6.00 | 6.00 | | | 6.00 | 7.50 | 7.50 | | | 6.00 |
| Ammonium Lauryl Sulfate (ALS) | 10.00 | 10.00 | | | 10.00 | 6.50 | 6.50 | 1.00 | | 10.00 |
| Sodium Laureth Sulfate (SE₃S) | | | 6.50 | 6.50 | | | | 6.00 | 6.00 | |
| Sodium Lauryl Sulfate (SLS) | | | 5.50 | 5.50 | | | | 7.00 | 10.00 | |
| Sodium Lauroamphoacetate⁽¹⁴⁾ | | | | | | | | | | |
| Cocaminopropionic Acid⁽¹⁵⁾ | | | | 1.00 | | | | | | |
| Cocamidopropyl Betaine⁽¹⁶⁾ | | | | 1.00 | | | | 2.00 | | |
| Cocamide MEA | | | 0.80 | 0.80 | | 0.80 | 0.80 | 0.85 | | |
| Cetyl Alcohol | | | 0.60 | 0.60 | | 0.60 | 0.60 | | | |
| Lauryl Alcohol | | | 0.35 | 0.35 | | | | | | |
| Laureth-4 Alcohol | 0.90 | 0.90 | | | 0.90 | | | | 0.90 | 0.90 |
| Dihydrogenated Tallowamidoethyl | | | | | | | | 0.15 | | |
| Hydroxyethylmonium Methosulfate⁽¹⁷⁾ | | | | | | | | | | |
| Trimethylammoniopropylmethacrylamide | | 0.50⁽²⁰⁾ | 0.10⁽²¹⁾ | 1.00⁽²⁰⁾ | 0.25⁽²¹⁾ | | | 0.05⁽²⁰⁾ | | 0.25⁽²⁵⁾ |
| chloride-N-Acrylamide copolymer^{(20,21,25)} | | | | | | | | | | |
| Trimethylammoniopropylmethacrylamide | | | | | | 0.40⁽²²⁾ | 0.10⁽²³⁾ | | | |
| chloride-N-vinylpyrrolidone | | | | | | | | | | |
| copolymer^{(22,23)} | | | | | | | | | | |
| Trimethylammoniopropylmethacrylamide | | | | | | | | | 0.40 | |
| chloride-N- | | | | | | | | | | |
| Methacrylamidopropyldimethylammonium | | | | | | | | | | |
| methylcarboxylate copolymer⁽²⁴⁾ | | | | | | | | | | |
| Trimethylammoniopropylmethacrylamide | 0.25 | | | | | | | | | |
| chloride-N-Behenyl | | | | | | | | | | |
| ethoxymethacrylate copolymer⁽²⁶⁾ | | | | | | | | | | |
| Ethylene Glycol Distearate | | | 1.50 | 1.50 | | 1.50 | 1.50 | 1.50 | 1.50 | |
| Trihydroxystearin⁽⁷⁾ | 0.10 | 0.10 | | | 0.10 | | | | | 0.10 |
| Polyethylene Glycol (14000)⁽⁸⁾ | | | 0.17 | 0.17 | | 0.17 | 0.17 | | | |
| Fragrance | 0.60 | 0.60 | 0.55 | 0.55 | 0.60 | 0.55 | 0.55 | 0.65 | 0.65 | 0.60 |
| Sodium Chloride | 0.40 | 0.40 | 0.80 | 0.80 | 0.40 | 0.80 | 0.80 | 1.40 | 1.40 | 0.40 |
| Ammonium Xylenesulfonate | | | | | | | | 0.20 | | |
| Citric Acid | 0.04 | 0.04 | 0.22 | 0.22 | 0.04 | 0.04 | 0.04 | 0.22 | 0.22 | 0.04 |
| Sodium Citrate | 0.40 | 0.40 | | | 0.40 | 0.40 | 0.40 | | | 0.40 |
| Sodium Benzoate | 0.25 | 0.25 | 0.25 | 0.25 | 0.25 | 0.25 | 0.25 | 0.25 | 0.25 | 0.25 |
| Ethylene Diamine Tetra Acetic Acid | 0.10 | 0.10 | 0.10 | 0.10 | 0.10 | 0.10 | 0.10 | 0.10 | 0.10 | 0.10 |
| Dimethicone^{(9,10,11)} | 1.00⁽⁹⁾ | 0.50⁽⁹⁾ | 0.50⁽⁹⁾ | 2.00⁽¹⁰⁾ | 1.00⁽⁹⁾ | 0.80⁽⁹⁾ | | 1.00⁽⁹⁾ | 2.00⁽⁹⁾ | 1.00⁽⁹⁾ |
| Polydecene⁽¹²⁾ | | | | | | | | 0.40 | | |
| Trimethylolpropane | | | | | | | | 0.10 | | |
| Tricaprylate/Tricaprate⁽¹³⁾ | | | | | | | | | | |
| Cosmetic Pigment (Mica, Titanium | | 0.10 | | | | | | | | |
| Dioxide)⁽¹⁹⁾ | | | | | | | | | | |
| Water and Minors (QS to 100%) | | | | | | | | | | |

Comparative Example

| | EXAMPLE COMPOSITION |
|---|---|
| | 21 |
| Ammonium Laureth Sulfate (AE₃S) | 6.00 |
| Ammonium Lauryl Sulfate (ALS) | 10.00 |
| Laureth-4 Alcohol | 0.90 |
| Trihydroxystearin⁽⁷⁾ | 0.10 |
| Fragrance | 0.60 |
| Sodium Chloride | 0.40 |
| Citric Acid | 0.04 |
| Sodium Citrate | 0.40 |
| Sodium Benzoate | 0.25 |
| Ethylene Diamine Tetra Acetic Acid | 0.10 |
| Dimethicone^{(9,10,11)} | 1.00⁽⁹⁾ |
| Water and Minors (QS to 100%) | |

⁽¹⁾HMW MAPTAC (Rhodia) [charge density = 4.5 meq/g, molecular weight ~860,000]
⁽²⁾HHMW MAPTAC (Rhodia) [charge density = 4.5 meq/g, molecular weight ~1,500,000]
⁽³⁾Diquat (Rhodia) [charge density = 5.6 meq/g, molecular weight ~252,000]
⁽⁴⁾DADMAC (Rhodia) [charge density = 6.2 meq/g, molecular weight ~1,200,000]
⁽⁵⁾DADMAC (Rhodia) [charge density = 6.2 meq/g, molecular weight ~175,000]
⁽⁶⁾Homopolymer of METAMS (Rhodia) [charge density = 3.5 meq/g, molecular weight ~313,000]
⁽⁷⁾Thixcin R (Rheox)
⁽⁸⁾PEG 14M (Dow Chemical)
⁽⁹⁾Viscasil 330M (Momentive)
⁽¹⁰⁾Dow Corning ® 1664 Emulsion (Dow Corning)
⁽¹¹⁾Dow Corning ® 2-1865 Microemulsion (Dow Corning)
⁽¹²⁾Puresyn 6, MCP-1812 (Mobil)
⁽¹³⁾Mobil P43 (Mobil)
⁽¹⁴⁾Miranol Ultra L32 (Rhodia)
⁽¹⁵⁾MACKAM 151C (McIntyre)
⁽¹⁶⁾Tegobetaine F-B (Goldschmidt)
⁽¹⁷⁾Varisoft 110 (Witco)
⁽¹⁸⁾Triqaut (Rhodia) [charge density = 6.07]
⁽¹⁹⁾Timiron MP-149 Diamond Cluster (EMD Chemicals)
⁽²⁰⁾1:9 AM:MAPTAC (Rhodia) [charge density = 4.4 meq/g, molecular weight ~1,250,000]
⁽²¹⁾3:7 AM:MAPTAC (Rhodia) [charge density = 4.0 meq/g, molecular weight ~500,000]
⁽²²⁾1:9 VP:MAPTAC (Rhodia) [charge density = 4.3 meq/g, molecular weight ~242,000]
⁽²³⁾3:7 VP:MAPTAC (Rhodia) [charge density = 3.7 meq/g, molecular weight ~503,000]
⁽²⁴⁾1:1 AP:MAPTAC (Rhodia) [charge density = 4.0 meq/g, molecular weight ~243,000]
⁽²⁵⁾5:5 AM:MAPTAC (Rhodia) [charge density = 3.4 meq/g, molecular weight ~500,000]
⁽²⁶⁾1:9 BEM:MAPTAC (Rhodia) [charge density = 2.6 meq/g]

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating damaged hair comprising the steps of:
a) contacting said damaged hair with a shampoo composition, said shampoo composition comprising:
i from about 5% to about 50% of an anionic surfactant;
ii from about 0.025% to about 5% by weight of a synthetic, non-crosslinked, cationic polymer having a cationic charge density of from about 2 meq/gm to about 7 meq/gm, wherein said synthetic cationic polymer forms lyotropic liquid crystals upon combination with said anionic surfactant; and
iii water; and
b) rinsing said composition from said hair.
wherein following said treatment, silicone deposition efficiency (DE) on said hair is greater than 1.

2. The method of claim 1, wherein the cationic charge density of said cationic polymer is from about 3 meq/gm to about 7 meq/gm.

3. The method of claim 1, wherein the cationic charge density of said cationic polymer is from about 4 meq/gm to about 7 meq/gm.

4. The method of claim 1, wherein said cationic polymer has an average molecular weight of from about 1,000 to about 5,000,000.

5. The method of claim 1, wherein said cationic polymer has an average molecular weight of from about 10,000 to about 2,000,000.

6. The method of claim 1, wherein said cationic polymer has an average molecular weight of from about 100,000 to about 2,000,000.

7. The method of claim 1, wherein said cationic polymer comprises monomers selected from the group consisting of dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide; ethylenimine, vinylamine, 2-vinylpyridine, 4-vinylpyridine, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride, diallyldimethyl ammonium chloride, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride and trimethyl ammonium propyl(meth)acrylamido chloride.

8. The method of claim 1, wherein said cationic polymer is diallyldimethyl ammonium chloride.

9. The method of claim 1, wherein said shampoo composition further comprises an ingredient selected from the group consisting of oily conditioning agents, hydrocarbon oils, polyolefins, fatty esters, fluorinated conditioning compounds, fatty alcohols, quaternary ammonium compounds, polyethylene glycols, anti-dandruff actives, anti-microbial actives, inorganic or synthetic particles, opacifying agents, suspending agents, propellants, paraffinic hydrocarbons, mono or divalent salts, fragrances, vitamins, chelating agents, colorants, pigments, dyes and mixtures thereof.

10. The method of claim 9, wherein said oily conditioning agent is polydimethylsiloxane.

11. The method of claim 1, wherein said damaged hair is chemically-treated hair.

12. The method of claim 1, wherein said damaged hair is mechanically damaged hair.

13. The method of claim 1, wherein said damaged hair is environmentally damaged hair.

14. An article of manufacture useful for treating damaged hair:
(a) a package;
(b) said package comprising a shampoo composition, wherein said shampoo composition comprises lyotropic liquid crystals; and
(c) information in association with said package comprising an instruction to use said shampoo composition to treat damaged hair.

15. An article of manufacture according to claim 14, wherein said information further comprises information which communicates to a consumer that the shampoo composition provides hair with increased silicone deposition efficiency.

16. An article of manufacture according to claim 14, wherein said information is indicia is selected from the group consisting of words, pictures, symbols, or mixtures thereof.

17. A method for promoting the sale of the article of claim 14, comprising the steps of:
(a) displaying the shampoo product in a retail store; and
(b) providing promotional materials to consumers;
wherein said promotional materials comprise information which communicates to a consumer that said shampoo composition's ability to treat damaged hair.

18. A method according to claim 17, wherein said promotional materials further comprise samples of said shampoo composition.

19. A method according to claim 17, wherein said promotional materials further comprise one or more discount coupons.

20. A method according to claim 17, wherein said promotional materials are further provided to hair styling salons.
